(19) United States
(12) Patent Application Publication
(10) Pub. No.: US 2013/0026132 A1
(43) Pub. Date: Jan. 31, 2013

(54) PLANARIZING FILM-FORMING COMPOSITION FOR HARD DISK AND HARD DISK PRODUCTION METHOD USING SAME

(71) Applicants: Taku Kato, Funabashi-shi (JP); Junpei Kobayashi, Funabashi-shi (JP); Keisuke Shuto, Funabashi-shi (JP); Masayoshi Suzuki, Funabashi-shi (JP)

(72) Inventors: Taku Kato, Funabashi-shi (JP); Junpei Kobayashi, Funabashi-shi (JP); Keisuke Shuto, Funabashi-shi (JP); Masayoshi Suzuki, Funabashi-shi (JP)

(73) Assignee: NISSAN CHEMICAL INDUSTRIES, LTD., Tokyo (JP)

(21) Appl. No.: 13/640,923

(22) PCT Filed: Apr. 18, 2011
(86) PCT No.: PCT/JP2011/059540

§ 371 Date: Oct. 12, 2012

(30) Foreign Application Priority Data
Apr. 22, 2010 (JP) 2010-099139

(51) Int. Cl. C08F 120/10; C07C 69/604; C08F 120/26; G11B 5/84

(52) U.S. Cl. 216/22; 560/201; 522/181; 560/194; 522/182

(57) ABSTRACT

A planarizing film-forming composition for a hard disk that is a non-magnetic filler is sufficiently filled into fine grooves on a magnetic material surface (surface), and is required not to cause contraction in the filled parts at the time of photo-curing (at the time of exposure) and post-exposure baking; and a method for producing a hard disk using the composition. The composition comprising at least one polyfunctional (meth)acrylate compound being in a liquid state at room temperature and atmospheric pressure and having a molecular weight of 300 to 10,000. The compound preferably has 2 to 20 (meth)acrylate groups in the molecule, or the compound preferably has a molecular weight of 300 to 2,300. A method for producing a hard disk comprising: forming a concave-convex shape on the surface; covering the surface having the concave-convex shape with the composition; and etching the covered surface for planarization until the surface is exposed.

### TECHNICAL FIELD

The present invention relates to a planarizing film-forming composition for a hard disk and a method for producing a hard disk using the composition.

### BACKGROUND ART

In hard disk drives, both head performance and magnetic recording medium performance have been developed and consequently the increase in capacity and the reduction in size have been developed.

From the viewpoint of an increase in performance of the magnetic recording medium, the increase in surface recording density has achieved the increase in the capacity. However, an increase in the recording density raises a problem of magnetic field expansion for the magnetic head. The magnetic field expansion could not be reduced more than a certain level even when a head size is reduced and thus a phenomenon, so-called side-writing, is caused. When the side-writing is caused, such side-writing causes erroneous writing on adjacent tracks during recording and may overwrite existing data to erase the data. In addition, the magnetic field expansion leads to reading of superfluous signals from adjacent tracks to cause crosstalk during reproduction.

In order to solve such a problem, there have been developed technologies of discrete track media and bit patterned media in which the space between tracks is filled with a non-magnetic material to thereby physically and magnetically separate the tracks.

As a method for forming a magnetic or non-magnetic pattern during the production of such a medium, application of photo-nanoimprinting has been developed (see Patent Document 1). When a medium is produced by applying the photo-nanoimprinting as above, a pattern is formed on a surface of a magnetic material by the photo-nanoimprinting, followed by dry etching to form grooves (concave-convex shape) on the surface of the magnetic material. Next, into the grooves, a non-magnetic material is embedded, followed by planarization to expose the surface of the magnetic material, thereby forming a plurality of tracks with the magnetic material and the non-magnetic material separated from each other. Then, the planarized surface of the magnetic material having the magnetic material areas and the non-magnetic material areas is covered with a hard substance (for example, diamond-like carbon) to form a face that is to be in contact with a magnetic head.

Meanwhile, as a related art, there has been developed a method using an acrylate compound as a surface protective layer for CDs, DVDs, and the like (see Patent Document 2).

In addition, in a magnetic recording medium including a non-magnetic material layer on a substrate, and a magnetic material layer on a top face of the non-magnetic material layer, in order to cure the non-magnetic material layer by irradiation with a smaller amount of electron beam, there has been developed a method using a non-magnetic material powder and an electron beam curable multifunctional acrylate monomer (see Patent Document 3).

### RELATED ART DOCUMENTS

### Patent Documents

Patent Document 1: Japanese Patent Application Publication No. JP-A-2010-037541 (JP 2010-037541 A)
Patent Document 2: Japanese Patent Application Publication No. JP-A-2006-102744 (JP 2006-102744 A)
Patent Document 3: Japanese Patent Application Publication No. JP-A-2006-202414 (JP 2006-202414 A)

### SUMMARY OF THE INVENTION

### Problem to be Solved by the Invention

Acrylate compounds have been used as a surface protective film for optical media.

However, such an acrylate compound has not been developed for applying the compound as a non-magnetic filler (planarizing film-forming composition) that is used in the technologies of the discrete track media and the bit patterned media, that is, in the method in which fine grooves (several tens of nanometers) are formed on a magnetic material surface, the grooves are filled with a non-magnetic material, followed by photo-curing for planarization to form tracks having magnetic material areas and non-magnetic material areas alternately.

In view of the above circumstances, the present invention provides a novel planarizing film-forming composition for a hard disk that is sufficiently filled into fine grooves on a magnetic material surface and is required not to cause contraction in the filled parts at the time of photo-curing (at the time of exposure) and at the time of post-exposure baking. The present invention also provides a method for producing a hard disk using the composition.

### Means for Solving the Problem

The present invention provides: as a first aspect, a planarizing film-forming composition for a hard disk that includes at least one polyfunctional (meth)acrylate compound being in a liquid state at room temperature and atmospheric pressure and having a molecular weight of 300 to 10,000;

as a second aspect, the planarizing film-forming composition for a hard disk according to the first aspect, in which the compound is a compound having 2 to 20 (meth)acrylate groups in the molecule;

as a third aspect, the planarizing film-forming composition for a hard disk according to the first aspect or the second aspect, in which the compound has a molecular weight of 300 to 2,300;

as a fourth aspect, the planarizing film-forming composition for a hard disk according to any one of the first aspect to the third aspect further including a photopolymerization initiator;

as a fifth aspect, a method for producing a hard disk that includes a first step of forming a concave-convex shape on a magnetic material surface, a second step of covering the magnetic material surface having the concave-convex shape with the planarizing film-forming composition as described in any one of the first aspect to the fourth aspect, and a third step of etching the covered magnetic material surface for planarization until the magnetic material surface is exposed;

as a sixth aspect, the method for producing a hard disk according to the fifth aspect further including a fourth step of covering the planarized magnetic material surface with a hard substance;

as a seventh aspect, the method for producing a hard disk according to the fifth aspect or the sixth aspect, in which in the first step, the concave-convex shape is formed by nanoimprinting;

as an eighth aspect, the method for producing a hard disk according to any one of the fifth aspect to the seventh aspect, in which in the third step, the planarization is performed by dry etching; and

as a ninth aspect, the method for producing a hard disk according to any one of the sixth aspect to the eighth aspect, in which the hard substance used in the fourth step is diamond-like carbon.

### Effects of the Invention

In the production of media, comparing with the case using a planarizing film-forming composition containing an inorganic compound as a composition to be filled into fine grooves on a magnetic material surface, the planarizing film-forming composition containing an organic compound for a hard disk of the present invention has advantageous effects as below.

When the planarizing film-forming composition containing an inorganic compound is used, the composition is applied onto a concave-convex substrate, the substrate is heated at a temperature of 200° C. or higher to perform reflow, the inorganic coating is scraped by CMP, wet etching, or the like, the substrate is heated to be cured and then is subjected to dry etching for surface planarization, and diamond-like carbon is applied onto the surface.

In contrast, when the planarizing film-forming composition containing an organic compound of the present invention is used, the composition is applied onto a concave-convex substrate, the substrate is subjected to exposure, then, if necessary, is subjected to post exposure bake, and then is subjected to dry etching for surface planarization, and diamond-like carbon is applied onto the surface. Therefore, when the planarizing film-forming composition containing an organic compound of the present invention is used, the composition can be filled into fine grooves on a magnetic material surface by coating alone without heating for reflow. That is, the planarizing film-forming composition of the present invention does not need the reflow in the coating process (in the second step of the present invention).

In addition, when the planarizing film-forming composition containing an inorganic compound is used, the inorganic coating is removed by CMP, wet etching, or the like, followed by dry etching for planarization, thereby increasing the number of steps.

In contrast, when the planarizing film-forming composition containing an organic compound of the present invention is used, the planarization can be performed by dry etching alone (in the third step of the present invention). Therefore, the simplified production process and the reduction in the number of steps can achieve productivity improvement and cost reduction.

### BRIEF DESCRIPTION OF THE DRAWINGS

FIG. 1 is a scanning electron microscope (SEM) photograph observing a cross section of a substrate made of silicon before the formation of a film and the substrate has a concave-convex shape that has a depth of 100 nm and in which lines and spaces are equally spaced by 30 nm.

FIG. 2 is a scanning electron microscope (SEM) photograph of the cross section of a substrate that was obtained by applying a planarizing film-forming composition prepared in Example 1 onto the substrate having a concave-convex shape in FIG. 1 and exposing the substrate followed by post exposure bake, for observing the flatness.

### MODES FOR CARRYING OUT THE INVENTION

The present invention is a planarizing film-forming composition for a hard disk that includes at least one polyfunctional (meth)acrylate compound being in a liquid state at room temperature and atmospheric pressure and having a molecular weight of 300 to 10,000.

The planarizing film-forming composition for a hard disk of the present invention includes the polyfunctional (meth)acrylate compound, a photopolymerization initiator, and an organic solvent and, if desired, may include an additive such as a surfactant.

In the present invention, the room temperature is 20 to 25° C.

The planarizing film-forming composition of the present invention has a solid content ratio of 0.01 to 20% by mass, 0.1 to 10% by mass, or 1 to 10% by mass. Here, the solid content means residual components of all components of the planarizing film-forming composition except organic solvents.

The planarizing film-forming composition of the present invention includes the polyfunctional (meth)acrylate compound in an amount of 50 to 99% by mass, 60 to 95% by mass, or 70 to 90% by mass, based on the amount of the solid content in the planarizing film-forming composition.

The planarizing film-forming composition of the present invention includes the photopolymerization initiator in an amount of 0.5 to 30% by mass, 5 to 30% by mass, or to 30% by mass, based on the amount of the solid content in the planarizing film-forming composition.

The planarizing film-forming composition of the present invention includes the additive such as a surfactant in an amount of 0.0001 to 1% by mass or 0.001 to 0.5% by mass, based on the amount of the solid content in the planarizing film-forming composition.

The planarizing film-forming composition of the present invention uses the polyfunctional (meth)acrylate compound as an organic compound. A (meth)acrylate compound having a small number of functional groups is unlikely to be contracted at the time of exposure, while a (meth)acrylate compound having a large number of functional groups is unlikely to be contracted at the time of post exposure bake after the exposure (the increase in cross-linking density can suppress heat contraction). From these points, the polyfunctional (meth)acrylate compound preferably has 2 to 20 (meth)acrylate groups in the molecule.

The polyfunctional (meth)acrylate compound preferably has a molecular weight of 300 to 2,300.

In the present invention, a (meth)acrylate compound means both an acrylate compound and a methacrylate compound and similarly, a (meth)acrylate group means both an acrylate group and a methacrylate group.

In the present invention, a polyfunctional (meth)acrylate compound has at least two acrylate groups, methacrylate groups, or a combination of them.

The polyfunctional (meth)acrylate compound can be exemplified by below compounds.

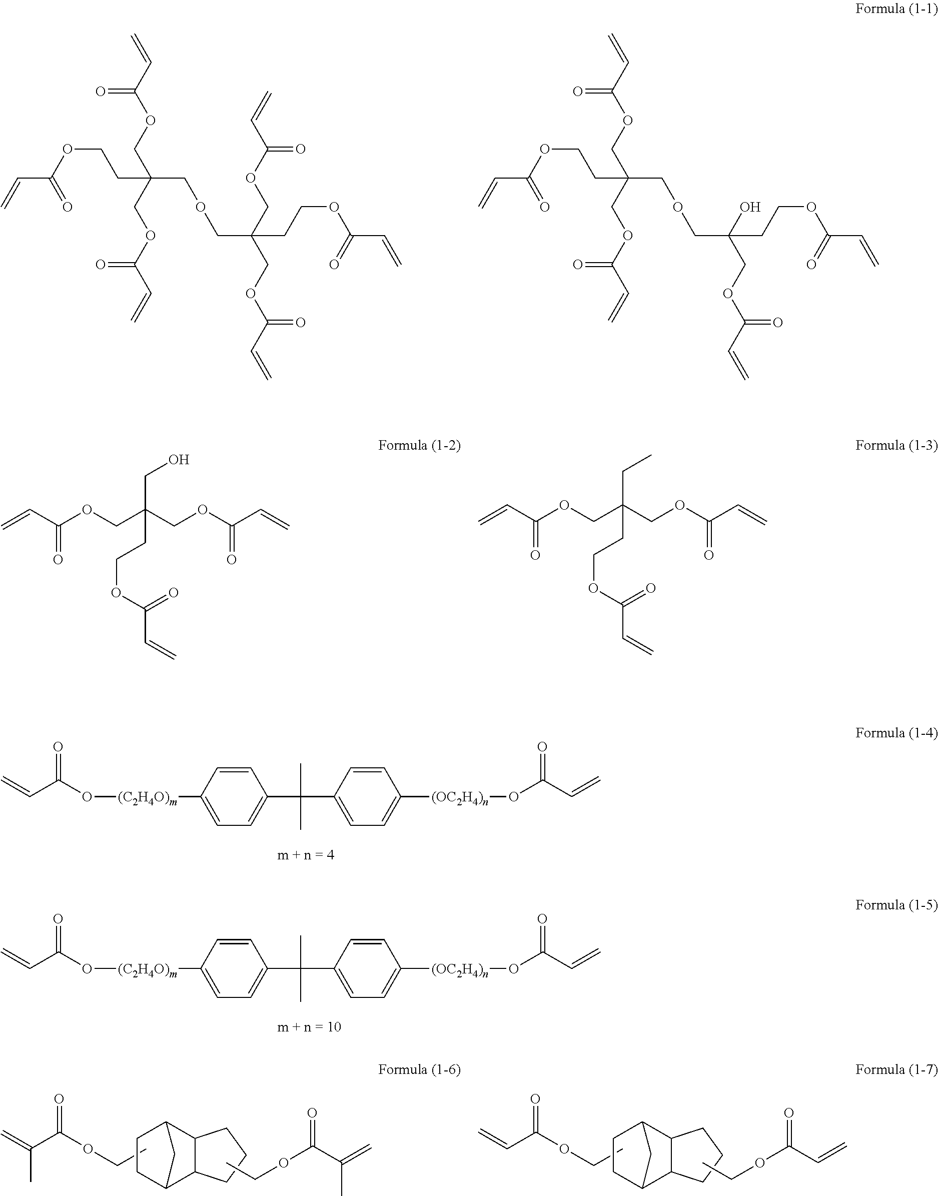

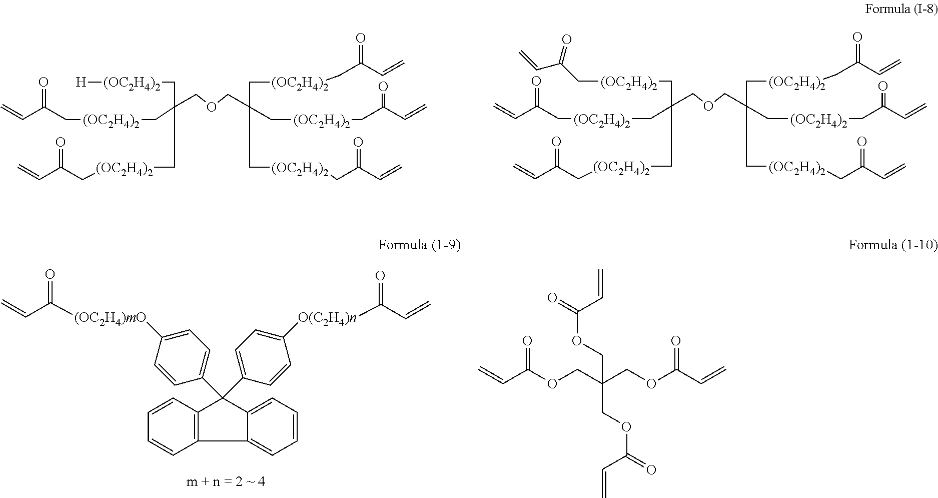

The multifunctional acrylate compound of Formula (1-1) includes a compound having a molecular weight of 552.57 and a compound having a molecular weight of 606.61 at a molar ratio of 50/50, is dipentaerythritol hexaacrylate that is liquid at room temperature (23° C.), and is available from Nippon Kayaku Co., Ltd.

The multifunctional acrylate compound of Formula (1-2) is pentaerythritol triacrylate having a molecular weight of 312.32 and being liquid at room temperature (23° C.) and is available from Sigma-Aldrich.

The multifunctional acrylate compound of Formula (1-3) is trimethylolpropane triacrylate having a molecular weight of 310.34 and being liquid at room temperature (23° C.) and is available from Shin Nakamura Chemical Co., Ltd.

The multifunctional acrylate compound of Formula (1-4) is ethoxylated bisphenol A diacrylate having a molecular weight of 400 and being liquid at room temperature (23° C.) and is available from Shin Nakamura Chemical Co., Ltd.

The multifunctional acrylate compound of Formula (1-5) is ethoxylated bisphenol A diacrylate having a molecular weight of 500 and being liquid at room temperature (23° C.) and is available from Shin Nakamura Chemical Co., Ltd.

The multifunctional methacrylate compound of Formula (1-6) is tricyclodecanedimethanol dimethacrylate having a molecular weight of 364.52 and being liquid at room temperature (23° C.) and is available from Shin Nakamura Chemical Co., Ltd.

The multifunctional acrylate compound of Formula (1-7) is tricyclodecanedimethanol diacrylate having a molecular weight of 336.47 and being liquid at room temperature (23° C.) and is available from Shin Nakamura Chemical Co., Ltd.

The multifunctional acrylate compound of Formula (1-8) is ethylene oxide modified dipentaerythritol hexaacrylate having a molecular weight of 500 and being liquid at room temperature (23° C.) and is available from Nippon Kayaku Co., Ltd.

The multifunctional acrylate compound of Formula (1-9) is bis-phenoxyethanol fluorene diacrylate having a molecular weight of 400 and being liquid at room temperature (23° C.) and is available from Osaka Gas Chemicals Co., Ltd.

The multifunctional acrylate compound of Formula (1-10) is pentaerythritol tetraacrylate having a molecular weight of 352.34 and being liquid at room temperature (23° C.) and is available from Shin Nakamura Chemical Co., Ltd.

These polyfunctional (meth)acrylate compounds may be used alone or as a mixture of two or more compounds.

The planarizing film-forming composition of the present invention may include a photopolymerization initiator. The photopolymerization initiator is not specifically limited as long as a compound can start polymerization of the polyfunctional (meth)acrylate compound by photoirradiation. As the photopolymerization initiator, for example, a compound that generates an acid (Bronsted acid or Lewis acid), a base, a radical, or a cation by photoirradiation can be used. Among them, in particular, a radical photopolymerization initiator is preferably used.

Examples of the radical photopolymerization initiator include trade name: IRGACURE [registered trademark] 369 (Formula 2-1, manufactured by BASF Japan Ltd., 2-benzyl-2-dimethylamino-1-(4-morpholinophenyl)-butanone-1), trade name: IRGACURE [registered trademark] 500 (Formula 2-2, manufactured by BASF Japan Ltd., 1-hydroxycyclohexyl phenyl ketone and benzophenone), trade name: IRGACURE [registered trademark] 819 (Formula 2-3, manufactured by BASF Japan Ltd., bis(2,4,6-trimethylbenzoin)-phenylphosphine oxide), trade name: IRGACURE [registered trademark] 651 (Formula 2-4, manufactured by BASF Japan Ltd., 2,2-dimethoxy-1,2-diphenylethan-1-one), trade name: IRGACURE [registered trademark] 184 (Formula 2-5, manufactured by BASF Japan Ltd., 1-hydroxycyclohexyl phenyl ketone), trade name: Darocur [registered trademark] 1173 (Formula 2-6, manufactured by BASF Japan Ltd., 2-hydroxy-2-methyl-1-phenylpropan-1-one), trade name: IRGACURE [registered trademark] 2959 (Formula 2-7, manufactured by BASF Japan Ltd., 1-(4-(2-hydroxyethoxy)phenyl)-2-hydroxy-2-methyl)-1-propan-1-one), trade name: IRGACURE [registered trademark] 127 (Formula 2-8, manufactured by BASF Japan Ltd., 2-hydroxy-1-(4-(4-(2-hydroxy-2-methylpropionyl)benzyl)phenyl)-2-methyl-propan-1-one), trade name: IRGACURE [registered trademark] 907 (Formula 2-9, manufactured by BASF Japan Ltd., 2-methyl-1-(4-methylthiophenyl)-2-morpholinopropan-1-one), trade name: IRGACURE [registered trademark] 379 (Formula 2-10, manufactured by BASF Japan Ltd., 2-(dimethylamino)-2-(4-(methylphenyl)methyl)-1-(4-(4-morpholinyl)phenyl)-1-butanone), and trade name: IRGACURE [registered trademark] OXE01 (Formula 2-11, manufactured by BASF Japan Ltd., 1,2-octanedione 1,4-(4-phenylthio)-2-(O-benzoyloxime)).

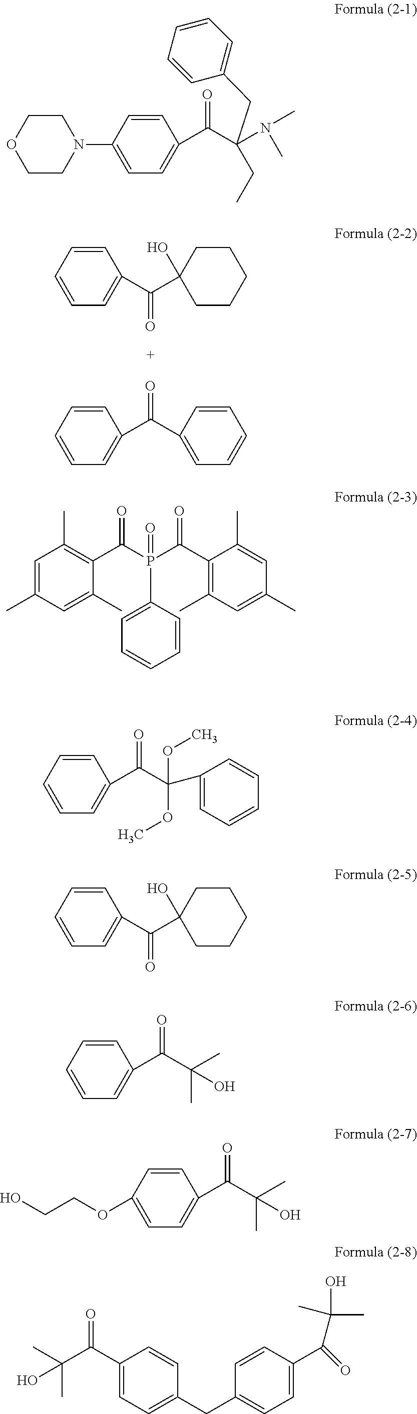

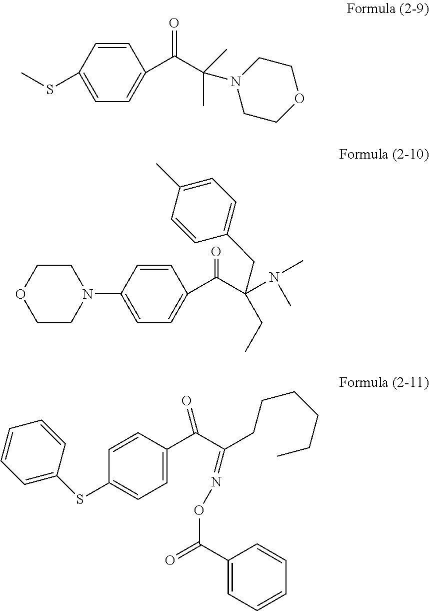 [SMILES: CSc1ccc(C(=O)C(C)(C)N2CCOCC2)cc1 ; CCC(Cc1ccc(C)cc1)(C(=O)c1ccc(N2CCOCC2)cc1)N(C)C ; CCCCCC/C(=N/OC(=O)c1ccccc1)C(=O)c1ccc(Sc2ccccc2)cc1]

The planarizing film-forming composition of the present invention may include a surfactant.

Examples of the surfactant include nonionic surfactants including polyoxyethylene alkyl ethers such as polyoxyethylene lauryl ether, polyoxyethylene stearyl ether, polyoxyethylene cetyl ether, and polyoxyethylene oleyl ether, polyoxyethylene alkylallyl ethers such as polyoxyethylene octylphenol ether and polyoxyethylene nonylphenol ether, polyoxyethylene/polyoxypropylene block copolymers, sorbitan fatty acid esters such as sorbitan monolaurate, sorbitan monopalmitate, sorbitan monostearate, sorbitan monooleate, sorbitan trioleate, and sorbitan tristearate, and polyoxyethylene sorbitan fatty acid esters such as polyoxyethylene sorbitan monolaurate, polyoxyethylene sorbitan monopalmitate, polyoxyethylene sorbitan monostearate, polyoxyethylene sorbitan trioleate, and polyoxyethylene sorbitan tristearate; fluorochemical surfactants including trade name: EFTOP [registered trademark] EF301, EF303, and EF352 (manufactured by Mitsubishi Materials Electronic Chemicals Co., Ltd. (formerly Tochem Products)), trade name: MEGAFAC [registered trademark] F171, F173, R-08, and R-30 (manufactured by DIC Corporation (formerly Dainippon Ink and Chemicals, Inc.)), Fluorad FC430 and FC431 (manufactured by Sumitomo 3M Ltd.), and trade name: Asahiguard [registered trademark] AG710 and Surflon [registered trademark] S-382, SC101, SC102, SC103, SC104, SC105, and SC106 (manufactured by Asahi Glass Co., Ltd.); and an organosiloxane polymer KP341 (manufactured by Shin-Etsu Chemical Co., Ltd.). These surfactants may be used alone or in combination of two or more of them.

The planarizing film-forming composition for a hard disk of the present invention may also include an organic solvent in addition to the polyfunctional (meth)acrylate compound and the photopolymerization initiator.

Examples of the organic solvent include toluene, p-xylene, o-xylene, styrene, ethylene glycol dimethyl ether, propylene glycol monomethyl ether, ethylene glycol monomethyl ether, propylene glycol, propylene glycol monoethyl ether, ethylene glycol monoethyl ether, ethylene glycol monoisopropyl ether, ethylene glycol methyl ether acetate, propylene glycol monomethyl ether acetate, ethylene glycol ethyl ether acetate, diethylene glycol dimethyl ether, propylene glycol monobutyl ether, ethylene glycol monobutyl ether, diethylene glycol diethyl ether, dipropylene glycol monomethyl ether, diethylene glycol monomethyl ether, dipropylene glycol monoethyl ether, diethylene glycol monoethyl ether, triethylene glycol dimethyl ether, diethylene glycol monoethyl ether acetate, diethylene glycol, 1-octanol, ethylene glycol, hexylene glycol, trimethylene glycol, 1-methoxy-2-butanol, cyclohexanol, diacetone alcohol, furfuryl alcohol, tetrahydrofurfuryl alcohol, propylene glycol, benzyl alcohol, 1,3-butanediol, 1,4-butanediol, 2,3-butanediol, γ-butyrolactone, acetone, methyl ethyl ketone, methyl isopropyl ketone, diethyl ketone, methyl isobutyl ketone, methyl n-butyl ketone, cyclohexanone, ethyl acetate, acetic isopropyl ketone, n-propyl acetate, isobutyl acetate, n-butyl acetate, methanol, ethanol, isopropanol, tert-butanol, allyl alcohol, n-propanol, 2-methyl-2-butanol, isobutanol, n-butanol, 2-methyl-1-butanol, 1-pentanol, 2-methyl-1-pentanol, 2-ethylhexanol, 1-octanol, ethylene glycol, hexylene glycol, trimethylene glycol, 1-methoxy-2-butanol, diacetone alcohol, furfuryl alcohol, tetrahydrofurfuryl alcohol, propylene glycol, benzyl alcohol, isopropyl ether, 1,4-dioxane, N,N-dimethylfomiamide, N,N-dimethylacetamide, N-methylpyrrolidone, 1,3-dimethyl-2-imidazolidinone, dimethyl sulfoxide, N-cyclohexyl-2-pyrrolidinone.

The present invention is a method for producing a hard disk. The method includes a first step of forming a concave-convex shape on a magnetic material surface, a second step of coating the magnetic material surface having the concave-convex shape with the planarizing film-forming composition of the present invention, and a third step of etching the covered magnetic material surface for planarization until the magnetic material surface is exposed.

In the first step, a concave-convex shape is formed on a magnetic material surface.

As a method for forming the concave-convex shape on the magnetic material surface, for example, a track pattern is formed on the magnetic material surface by photo- or thermal-nanoimprinting. Next, the magnetic material surface is processed using the pattern by dry etching to form the concave-convex shape. As a gas used for the etching, a fluorine-containing gas is preferably used to perform the dry etching. Examples of the fluorine-containing gas include tetrafluoromethane (CF₄), perfluorocyclobutane (C₄F₈), perfluoropropane (C₃F₈), trifluoromethane, and difluoromethane (CH₂F₂).

In the second step, onto the magnetic material surface on which the concave-convex shape has been formed, the planarizing film-forming composition of the present invention is applied for coating. In the step, the planarizing film-forming composition is applied onto the magnetic material having the concave-convex face, the concave-convex face is filled with the planarizing film-forming composition, and the concave-convex face may be further overcoated with the planarizing film-forming composition. Then, the planarizing film-forming composition is cured by photoirradiation.

Usable examples of the coating method include spin coating, dip coating, flow coating, ink-jetting, spraying, bar coating, gravure coating, roll coating, transfer printing, brush coating, blade coating, and air knife coating.

In the dip coating, an organic solvent containing methyl nonafluoroisobutyl ether (containing methyl nonafluorobutyl ether as an isomer) or 1,1,1,2,2,3,4,5,5,5-decafluoropentane may be used.

The spin coating is preferably used. For example, the coating can be performed at a rotation speed of 10 to 10,000 rpm for 3 to 60 seconds.

The film thickness can be within a range from 5 nm to 10 μm and particularly within a range from 5 nm to 100 nm because the concave-convex shape has a size of several tens of nanometers.

The photoirradiation can be performed, for example, using light having a wavelength of 150 nm to 1,000 nm, 200 to 700 nm, or 300 to 600 nm. The photoirradiation can be performed, for example, using an extra-high pressure mercury lamp, a flash UV lamp, a high pressure mercury lamp, a low pressure mercury lamp, a DEEP-UV (deep ultraviolet) lamp, a xenon short arc lamp, a short arc metal halide lamp, a YAG laser excitation lamp, or a xenon flash lamp. The photoirradiation can be performed, for example, using an extra-high pressure mercury lamp by applying light of any wavelength from about 250 nm to about 650 nm including emission line spectra having a wavelength peak of 289 nm, 297 nm, 303 nm, 313 nm (j-ray), 334 nm, or 365 nm (i-ray) in an ultraviolet light region or 405 nm (h-ray), 436 nm (g-ray), 546 nm, or 579 nm in a visible light region. The amount of irradiation is 10 to 1,000 mW or 10 to 100 mW and the irradiation is performed for 2 to 100 seconds or 5 to 20 seconds.

In the second step, after the exposure, post exposure bake may be performed as necessary. The post exposure bake is performed in a condition appropriately selected from a heating temperature of 70° C. to 170° C. and a heating time of 1 to 10 minutes.

In the third step, the magnetic material surface coated with the planarizing film is etched until the magnetic material surface is exposed, thereby planarizing the magnetic material surface and the planarizing film surface.

The etching is performed using an etching gas such as tetrafluoromethane (CF₄), perfluorocyclobutane (C₄F₈), perfluoropropane (C₃F₈), trifluoromethane, carbon monoxide, argon, oxygen, nitrogen, sulfur hexafluoride, difluoromethane, nitrogen trifluoride, and chlorine trifluoride.

In this manner, on the magnetic material surface, grooves of the non-magnetic material (grooves filled with the planarizing film of the present invention) are formed as the track pattern and the magnetic material surface and the non-magnetic material surface form a planar face.

In the present invention, as a fourth step, the planarized magnetic material surface may be coated with a hard substance having a thickness of several tens of nanometers by deposition. Examples of the hard substance include diamond-like carbon.

### EXAMPLES

The present invention will be described hereinafter in further detail with reference to examples and comparative examples, but the present invention is not limited to the examples below. Each measurement equipment used in the examples is as below.

A film thickness was determined using an optical film thickness meter (F20 manufactured by Filmetrics).

The atomic force microscope used was NANOSCOPE IV Dimension 3100 manufactured by Veeco-Digital Instruments Inc. and an analysis was carried out using a single-crystal Si probe (Super-Sharp Type (Nano World)) at a spring constant of about 30 N/m and a resonance frequency of about 290 kHz.

The UV irradiation apparatus used was an electrodeless lamp system QRE 4016 manufactured by Ore Manufacturing Co., Ltd. and was used at an illumination intensity of 20 mW/cm².

### Example 1

Into a 30-mL egg-plant type flask, 2.0000 g of a 30% by mass solution of a mixture of dipentaerythritol hexaacrylate (manufactured by Nippon Kayaku Co. Ltd., trade name: KAYARAD DPHA (hereinafter, abbreviated as DPHA), Formula (1-1)) that is in a liquid form at 23° C. under atmospheric pressure and has a molecular weight of 500 to 650 g/moL in propylene glycol monomethyl ether acetate (hereinafter, abbreviated as PGMEA) was weighed and charged, 0.1200 g of IRGACURE [registered trademark] 369 (manufactured by BASF Japan Ltd., Formula (2-1)) was added, 0.0600 g of a 0.1% by mass solution of MEGAFAC [registered trademark] R-30 (manufactured by DIC Corporation) in PGMEA was added, 12.8215 g of PGMEA was added, and then the whole was stirred using a stirrer at room temperature for 24 hours to prepare a uniform transparent solution (ASV-1).

### Example 2

A uniform transparent solution (ASV-2) was prepared in the same manner as in Example 1 except that the multifunctional acrylate compound used was pentaerythritol triacrylate (manufactured by Sigma-Aldrich, (hereinafter, abbreviated as PTA), Formula (1-2)) that is in a liquid form at 23° C. under atmospheric pressure and has a molecular weight of 312.32 g/moL, instead of DPHA.

### Example 3

A uniform transparent solution (ASV-3) was prepared in the same manner as in Example 1 except that the multifunctional acrylate compound used was trimethylolpropane triacrylate (manufactured by Shin Nakamura Chemical Co., Ltd., trade name: A-TMPT (hereinafter, abbreviated as A-TMPT), Formula (1-3)) that is in a liquid form at 23° C. under atmospheric pressure and has a molecular weight of 310.34 g/moL, instead of DPHA.

### Example 4

A uniform transparent solution (ASV-4) was prepared in the same manner as in Example 1 except that the multifunctional acrylate compound used was ethoxylated bisphenol A diacrylate (manufactured by Shin Nakamura Chemical Co., Ltd., trade name: A-BPE-4 (hereinafter, abbreviated as A-BPE-4), Formula (1-4)) that is in a liquid form at 23° C. under atmospheric pressure and has a molecular weight of about 400 g/moL, instead of DPHA.

### Example 5

A uniform transparent solution (ASV-5) was prepared in the same manner as in Example 1 except that the multifunctional acrylate compound used was ethoxylated bisphenol A diacrylate (manufactured by Shin Nakamura Chemical Co., Ltd., trade name: A-BPE-10 (hereinafter, abbreviated as A-BPE-10), Formula (1-5)) that is in a liquid form at 23° C. under atmospheric pressure and has a molecular weight of about 500 g/moL, instead of DPHA.

### Example 6

A uniform transparent solution (ASV-6) was prepared in the same manner as in Example 1 except that the multifunctional methacrylate compound used was tricyclodecanedimethanol dimethacrylate (manufactured by Shin Nakamura Chemical Co., Ltd., trade name: DCP (hereinafter, abbreviated as DCP), Formula (1-6)) that is in a liquid form at 23° C. under atmospheric pressure and has a molecular weight of 364.52 g/moL, instead of DPHA.

### Example 7

A uniform transparent solution (ASV-7) was prepared in the same manner as in Example 1 except that the multifunctional acrylate compound used was tricyclodecanedimethanol diacrylate (manufactured by Shin Nakamura Chemical Co., Ltd., trade name: A-DCP (hereinafter, abbreviated as A-DCP), Formula (1-7)) that is in a liquid form at 23° C. under atmospheric pressure and has a molecular weight of 336.47 g/moL, instead of DPHA.

### Example 8

A uniform transparent solution (ASV-8) was prepared in the same manner as in Example 1 except that the multifunctional acrylate compound used was ethylene oxide modified dipentaerythritol hexaacrylate (manufactured by Shin Nakamura Chemical Co., Ltd., trade name: DPEA-12 (hereinafter, abbreviated as DPEA-12), Formula (1-8)) that is in a liquid form at 23° C. under atmospheric pressure and has a molecular weight of about 500 g/moL, instead of DPHA.

### Example 9

A uniform transparent solution (ASV-9) was prepared in the same manner as in Example 1 except that the multifunctional acrylate compound used was a mixture of multifunctional urethane acrylates (manufactured by Shin Nakamura Chemical Co., Ltd., trade name: UA-53H (hereinafter, abbreviated as UA-53H)) that is in a liquid form at 23° C. under atmospheric pressure and has a molecular weight of 2,300 g/moL and a number of functional groups of 15, instead of DPHA.

### Example 10

A uniform transparent solution (ASV-10) was prepared in the same manner as in Example 1 except that the multifunctional acrylate compound used was bis-phenoxyethanol fluorene diacrylate (manufactured by Osaka Gas Chemicals Co., Ltd., trade name: EA0200 (hereinafter, abbreviated as EA0200), Formula (1-9)) that is in a liquid form at 23° C. under atmospheric pressure and has a molecular weight of about 400 g/moL, instead of DPHA.

### Example 11

A uniform transparent solution (ASV-11) was prepared in the same manner as in Example 1 except that the multifunctional acrylate compound used was pentaerythritol tetraacrylate (manufactured by Shin Nakamura Chemical Co., Ltd., trade name: A-TMMT (hereinafter, abbreviated as A-TMMT), Formula (1-10)) that is in a liquid form at 23° C. under atmospheric pressure and has a molecular weight of 352.34 g/moL, instead of DPHA.

### Example 12

Into a 30-mL egg-plant type flask, 2.0000 g of a 30% by mass solution of DPHA (Formula (1-1)) that is in a liquid form at 23° C. under atmospheric pressure in PGMEA and 0.5000 g of a 30% by mass solution of EA0200 (Formula (1-9)) that is in a liquid form at 23° C. under atmospheric pressure in PGMEA were added, 0.1500 g of IRGACURE [registered trademark] 369 was added, 0.0750 g of a 0.1% by mass solution of MEGAFAC [registered trademark] R-30 in PGMEA was added, 19.7769 g of PGMEA was added, and then the whole was stirred using a stirrer at room temperature for 24 hours to prepare a uniform transparent solution (ASV-12).

### Example 13

Into a 30-mL egg-plant type flask, 1.0000 g of a 30% by mass solution of DPHA (Formula (1-1)) that is in a liquid form at 23° C. under atmospheric pressure in PGMEA and 1.0000 g of a 30% by mass solution of EA0200 (Formula (1-9)) that is in a liquid form at 23° C. under atmospheric pressure in PGMEA were added, 0.1200 g of IRGACURE [registered trademark] 369 was added, 0.0600 g of a 0.1% by mass solution of MEGAFAC [registered trademark] R-30 in PGMEA was added, 12.8215 g of PGMEA was added, and then the whole was stirred using a stirrer at room temperature for 24 hours to prepare a uniform transparent solution (ASV-13).

### Example 14

Into a 30-mL egg-plant type flask, 0.500 g of a 30% by mass solution of DPHA

(Formula (1-1)) that is in a liquid form at 23° C. under atmospheric pressure in PGMEA and 2.0000 g of a 30% by mass solution of EA0200 (Formula (1-9)) that is in a liquid form at 23° C. under atmospheric pressure in PGMEA were added, 0.1500 g of IRGACURE [registered trademark] 369 was added, 0.0750 g of a 0.1% by mass solution of MEGAFAC [registered trademark] R-30 in PGMEA was added, 19.7769 g of PGMEA was added, and then the whole was stirred using a stirrer at room temperature for 24 hours to prepare a uniform transparent solution (ASV-14).

### Comparative Example 1

A uniform transparent solution (ASV-15) was prepared in the same manner as in Example 1 except that the multifunctional acrylate compound used was neopentyl glycol diacrylate (manufactured by Shin Nakamura Chemical Co., Ltd., (hereinafter, abbreviated as NPGDA), Formula (3-1)) that is in a liquid form at 23° C. under atmospheric pressure and has a molecular weight of 212.24 g/moL, instead of DPHA.

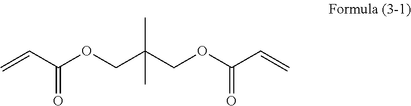

### Comparative Example 2

A uniform transparent solution (ASV-16) was prepared in the same manner as in Example 1 except that the multifunctional acrylate compound used was methyl methacrylate ((hereinafter, abbreviated as MMA), Formula (3-2)) that is in a liquid form at 23° C. under atmospheric pressure and has a molecular weight of 100.12 g/moL, instead of DPHA.

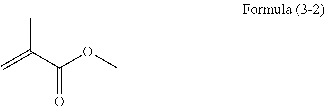

### Comparative Example 3

A uniform transparent solution (ASV-17) was prepared in the same manner as in Example 1 except that the multifunctional acrylate compound used was ethoxylated trisacryl isocyanulate (manufactured by Shin Nakamura Chemical Co., Ltd., trade name: A-9300 (hereinafter, abbreviated as A-9300), Formula (3-3)) that is in a solid form at 23° C. under atmospheric pressure and has a molecular weight of 381.42 g/moL, instead of DPHA.

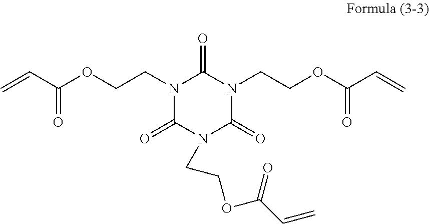 [SMILES: C=CC(=O)OCCn1c(=O)n(CCOC(=O)C=C)c(=O)n(CCOC(=O)C=C)c1=O]

### Comparative Example 4

Into a 30-mL egg-plant type flask, 3.0000 g of a 30% by mass solution of phenylsilsesquioxane (manufactured by Gelest; Mw 700, (hereinafter, abbreviated as PSQ), Formula (3-4)) that is in a solid form at 23° C. under atmospheric pressure in PGMEA and 0.0900 g of a 0.1% solution of MEGAFAC [registered trademark] R-30 (0.01 parts by mass based on 100 parts by mass of solid content) in PGMEA were added, 19.4123 g of PGMEA was added, and then the whole was stirred using a stirrer at room temperature for 24 hours to prepare a uniform transparent solution (ASV-18).

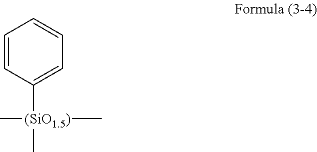

### Example 15

A uniform transparent solution (ASV-19) was prepared in the same manner as in Example 1 except that the radical photopolymerization initiator used was IRGACURE [registered trademark] OXE01 (manufactured by BASF Japan Ltd., Formula (2-11)), instead of IRGACURE [registered trademark] 369.

### Example 16

A uniform transparent solution (ASV-20) was prepared in the same manner as in Example 2 except that the radical photopolymerization initiator used was IRGACURE [registered trademark] OXE01 (manufactured by BASF Japan Ltd., Formula (2-11)), instead of IRGACURE [registered trademark] 369.

### Example 17

A uniform transparent solution (ASV-21) was prepared in the same manner as in Example 3 except that the radical photopolymerization initiator used was IRGACURE [registered trademark] OXE01 (manufactured by BASF Japan Ltd., Formula (2-11)), instead of IRGACURE [registered trademark] 369.

### Example 18

A uniform transparent solution (ASV-22) was prepared in the same manner as in Example 4 except that the radical photopolymerization initiator used was IRGACURE [registered trademark] OXE01 (manufactured by BASF Japan Ltd., Formula (2-11)), instead of IRGACURE [registered trademark] 369.

### Example 19

A uniform transparent solution (ASV-23) was prepared in the same manner as in Example 5 except that the radical photopolymerization initiator used was IRGACURE [registered trademark] OXE01 (manufactured by BASF Japan Ltd., Formula (2-11)), instead of IRGACURE [registered trademark] 369.

### Example 20

A uniform transparent solution (ASV-24) was prepared in the same manner as in Example 6 except that the radical photopolymerization initiator used was IRGACURE [registered trademark] OXE01 (manufactured by BASF Japan Ltd., Formula (2-11)), instead of IRGACURE [registered trademark] 369.

### Example 21

A uniform transparent solution (ASV-25) was prepared in the same manner as in Example 7 except that the radical photopolymerization initiator used was IRGACURE [registered trademark] OXE01 (manufactured by BASF Japan Ltd., Formula (2-11)), instead of IRGACURE [registered trademark] 369.

### Example 22

A uniform transparent solution (ASV-26) was prepared in the same manner as in Example 11 except that the radical photopolymerization initiator used was IRGACURE [registered trademark] OXE01 (manufactured by BASF Japan Ltd., Formula (2-11)), instead of IRGACURE [registered trademark] 369.

### Comparative Example 5

A uniform transparent solution (ASV-27) was prepared in the same manner as in Comparative Example 1 except that the radical photopolymerization initiator used was IRGACURE [registered trademark] OXE01 (manufactured by BASF Japan Ltd., Formula (2-11)), instead of IRGACURE [registered trademark] 369.

### Comparative Example 6

A uniform transparent solution (ASV-28) was prepared in the same manner as in Comparative Example 2 except that the radical photopolymerization initiator used was IRGACURE [registered trademark] OXE01 (manufactured by BASF Japan Ltd., Formula (2-11)), instead of IRGACURE [registered trademark] 369.

### Comparative Example 7

A uniform transparent solution (ASV-29) was prepared in the same manner as in Comparative Example 3 except that the radical photopolymerization initiator used was IRGACURE [registered trademark] OXE01 (manufactured by BASF Japan Ltd., Formula (2-11)), instead of IRGACURE [registered trademark] 369.

### Example 23

Into a 30-mL egg-plant type flask, 2.0000 g of a 30% by mass solution of DPHA (Formula (1-1)) that is in a liquid form at 23° C. under atmospheric pressure and has a molecular weight of 500 to 650 g/moL in PGMEA was weighed and charged, 0.0600 g of IRGACURE [registered trademark] 369 (manufactured by BASF Japan Ltd., Formula (2-1)) was added, 0.0600 g of a 0.1% by mass solution of MEGAFAC [registered trademark] R-30 (manufactured by DIC Corporation) in PGMEA was added, 14.3815 g of PGMEA was added, and then the whole was stirred using a stirrer at room temperature for 24 hours to prepare a uniform transparent solution (ASV-30).

### Example 24

Into a 30-mL egg-plant type flask, 2.0000 g of a 30% by mass solution of DPHA (Formula (1-1)) that is in a liquid form at 23° C. under atmospheric pressure and has a molecular weight of 500 to 650 g/moL in PGMEA was weighed and charged, 0.1800 g of IRGACURE [registered trademark] 369 (manufactured by BASF Japan Ltd., Formula (2-1)) was added, 0.0600 g of a 0.1% by mass solution of MEGAFAC [registered trademark] R-30 (manufactured by DIC Corporation) in PGMEA was added, 17.2615 g of PGMEA was added, and then the whole was stirred using a stirrer at room temperature for 24 hours to prepare a uniform transparent solution (ASV-31).

### Example 25

Into a 30-mL egg-plant type flask, 2.0000 g of a 30% by mass solution of DPHA (Formula (1-1)) that is in a liquid form at 23° C. under atmospheric pressure and has a molecular weight of 500 to 650 g/moL in PGMEA was weighed and charged, 0.1200 g of IRGACURE [registered trademark] 369 (manufactured by BASF Japan Ltd., Formula (2-1)) was added, 0.0600 g of a 0.1% by mass solution of MEGAFAC [registered trademark] R-30 (manufactured by DIC Corporation) in PGMEA was added, 12.3652 g of PGMEA was added, 3.4563 g of methanol was added, and then the whole was stirred using a stirrer at room temperature for 24 hours to prepare a uniform transparent solution (ASV-32).

### Example 26

Into a 30-mL egg-plant type flask, 2.0000 g of a 30% by mass solution of DPHA (Formula (1-1)) that is in a liquid form at 23° C. under atmospheric pressure and has a molecular weight of 500 to 650 g/moL in PGMEA was weighed and charged, 0.1200 g of IRGACURE [registered trademark] 369 (manufactured by BASF Japan Ltd., Formula (2-1)) was added, 0.0600 g of a 0.1% by mass solution of MEGAFAC [registered trademark] R-30 (manufactured by DIC Corporation) in PGMEA was added, 12.3652 g of PGMEA was added, 3.4563 g of isopropanol was added, and then the whole was stirred using a stirrer at room temperature for 24 hours to prepare a uniform transparent solution (ASV-33).

### Example 27

Into a 30-mL egg-plant type flask, 2.0000 g of a 30% by mass solution of DPHA (Formula (1-1)) that is in a liquid form at 23° C. under atmospheric pressure and has a molecular weight of 500 to 650 g/moL in PGMEA was weighed and charged, 0.1200 g of IRGACURE [registered trademark] 369 (manufactured by BASF Japan Ltd., Formula (2-1)) was added, 0.0600 g of a 0.1% by mass solution of MEGAFAC [registered trademark] R-30 (manufactured by DIC Corporation) in PGMEA was added, 12.3652 g of PGMEA was added, 3.4563 g of anisole was added, and then the whole was stirred using a stirrer at room temperature for 24 hours to prepare a uniform transparent solution (ASV-34).

<Film Formation of Planarizing Film-Forming Composition and Volume Contraction Ratio>

Each planarizing film-forming composition of ASV-1 to ASV-17 and ASV-19 to ASV-34 was applied onto a silicon wafer having a size of 4 inch by spin coating so as to have a film thickness of 85 nm. The film thickness was determined with an optical film thickness meter (the film thickness after spin coating is regarded as X). The film after spin coating was exposed to light using an UV irradiation apparatus (wavelength 365 nm) at an illumination intensity of 20 mW/cm² for 10 seconds under atmospheric pressure. The film thickness after exposure was determined with an optical film thickness meter (the film thickness after exposure is regarded as Y). Next, the film was subjected to post-exposure bake under atmospheric pressure using a hot plate at 130° C. for 5 minutes. The film thickness after baking was determined with an optical film thickness meter (the film thickness after baking is regarded as Z).

The volume contraction ratio at the time of exposure is defined as the value that is obtained by the subtraction of the value of Y expressed in percentage from 100 where the film thickness X is 100%.

The volume contraction ratio at the time of baking is defined as the value that is obtained by the subtraction of the value of Z expressed in percentage from 100 where the film thickness Y is 100%.

The planarizing film-forming composition of ASV-18 was applied onto a silicon wafer having a size of 4 inch by spin coating so as to have a film thickness of 85 nm. The film thickness determined with an optical film thickness meter was 85.0 nm (the film thickness after spin coating is regarded as L). The film after spin coating was baked under atmospheric pressure using a hot plate at 130° C. for 5 minutes. The film thickness after baking determined with an optical film thickness meter was 85.3 nm (the film thickness after baking is regarded as M).

The volume contraction ratio at the time of baking is defined as the value that is obtained by the subtraction of the value of M expressed in percentage from 100 where the film thickness L is 100%.

TABLE 1
Volume Contraction Ratio

| | | Volume contraction |
|---|---|---|
| | Volume contraction | ratio after exposure |
| | ratio after exposure | and baking |
| Film of ASV-1 (Example 1) | 5.2% | 1.3% |
| Film of ASV-2 (Example 2) | 6.5% | 1.5% |
| Film of ASV-3 (Example 3) | 6.7% | 2.2% |
| Film of ASV-4 (Example 4) | 3.7% | 3.8% |
| Film of ASV-5 (Example 5) | 5.9% | 2.4% |
| Film of ASV-6 (Example 6) | 4.4% | 4.8% |
| Film of ASV-7 (Example 7) | 4.8% | 3.3% |
| Film of ASV-8 (Example 8) | 3.7% | 5.7% |
| Film of ASV-9 (Example 9) | 4.6% | 1.6% |
| Film of ASV-10 (Example 10) | 1.2% | 3.2% |
| Film of ASV-11 (Example 11) | 5.1% | 4.1% |
| Film of ASV-12 (Example 12) | 4.5% | 1.5% |
| Film of ASV-13 (Example 13) | 3.2% | 1.6% |
| Film of ASV-14 (Example 14) | 2.2% | 2.8% |
| Film of ASV-15 (Comparative | 25.8% | 12.8% |
| Example 1) | | |
| Film of ASV-16 (Comparative | 40.7% | 79.9% |
| Example 2) | | |
| Film of ASV-17 (Comparative | 4.0% | 5.5% |
| Example 3) | | |

TABLE 2
Volume Contraction Ratio

| | Volume contraction ratio after baking |
|---|---|
| Film of ASV-18 (Comparative | −0.3% |
| Example 4) | |

TABLE 3
Volume Contraction Ratio

| | | Volume contraction |
|---|---|---|
| | Volume contraction | ratio after exposure |
| | ratio after exposure | and baking |
| Film of ASV-19 (Example 15) | 6.5% | 2.7% |
| Film of ASV-20 (Example 16) | 8.4% | 3.6% |
| Film of ASV-21 (Example 17) | 9.2% | 6.5% |
| Film of ASV-22 (Example 18) | 5.2% | 7.5% |
| Film of ASV-23 (Example 19) | 6.2% | 7.3% |
| Film of ASV-24 (Example 20) | 6.4% | 6.9% |
| Film of ASV-25 (Example 21) | 7.8% | 7.1% |
| Film of ASV-26 (Example 22) | 6.8% | 7.1% |
| Film of ASV-27 (Comparative | 48.5% | 17.9% |
| Example 5) | | |
| Film of ASV-28 (Comparative | 50.6% | 88.8% |
| Example 6) | | |
| Film of ASV-29 (Comparative | 5.6% | 6.6% |
| Example 7) | | |
| Film of ASV-30 (Example 23) | 5.4% | 1.0% |
| Film of ASV-31 (Example 24) | 5.1% | 1.6% |
| Film of ASV-32 (Example 25) | 5.2% | 1.3% |
| Film of ASV-33 (Example 26) | 5.2% | 1.3% |
| Film of ASV-34 (Example 27) | 5.2% | 1.4% |

In the volume contraction ratio measurement, it is considered that a film having a ratio within about 10% is planarized.

The results of the films of ASV-1 to ASV-14 and ASV-15 to ASV-17 revealed that a film using a polyfunctional (meth)acrylate compound having a molecular weight of 300 or less remarkably increased the volume contraction ratios at the time of exposure and at the time of baking to have poor film reduction characteristics. When a composition having a large volume contraction ratio and a large film reduction is used for a planarizing film on HDD as a material for embedding level difference, it is considered to readily reflect the shape of the level difference to deteriorate the flatness. The deterioration of the flatness leads to the loss of smoothness on HDD, and a magnetic head crashes the substrate surface to interfere with normal operation.

The results of the films of ASV-19 to ASV-26 and ASV-27 to ASV-29 revealed that, even when radical photopolymerization initiators were changed, a film using a polyfunctional (meth)acrylate compound having a molecular weight of 300 or less remarkably increased the volume contraction ratios at the time of exposure and at the time of baking to have poor film reduction characteristics.

The comparison of the films of ASV-1, ASV-30, and ASV-31 revealed that, even when the amount of a radical photopolymerization initiator was changed within a range of 10 to 30 parts by mass with respect to the solid content of the planarizing film-forming composition, the volume contraction ratio was unlikely to be changed and was good.

The comparison of the films of ASV-1 and ASV-32 to ASV-34 revealed that, even when a mixed solvent was used in the planarizing film-forming composition of the present invention, the volume contraction ratio was unlikely to be changed and was good.

In addition, the film of ASV-18, which was polysiloxane, caused volume expansion at the time of thermal curing, thereby disabling the planarization.

<Film Formation of Planarizing Film-Forming Composition and Volume Contraction Ratio 2>

The planarizing film-forming composition of ASV-1 was applied onto a silicon wafer having a size of 4 inch by spin coating so as to have a film thickness of 85 nm. The film thickness was determined with an optical film thickness meter (the film thickness after spin coating is regarded as X). The film after spin coating was exposed to light using an UV irradiation apparatus (wavelength 365 nm) at an illumination intensity of 20 mW/cm² for 10 seconds under atmospheric pressure. The film thickness after exposure was determined with an optical film thickness meter (the film thickness after exposure is regarded as Y). Next, the film was subjected to post-exposure bake under atmospheric pressure using a hot plate at 160° C. for 5 minutes. The film thickness after baking was determined with an optical film thickness meter (the film thickness after baking is regarded as Z).

The volume contraction ratio at the time of exposure is defined as the value that is obtained by the subtraction of the value of Y expressed in percentage from 100 where the film thickness X is 100%.

The volume contraction ratio at the time of baking is defined as the value that is obtained by the subtraction of the value of Z expressed in percentage from 100 where the film thickness Y is 100%.

TABLE 4
Volume Contraction Ratio

| | Volume contraction ratio | Volume contraction ratio |
|---|---|---|
| | after exposure | after exposure and baking |
| Film of ASV-1 | 5.2% | 2.2% |
| (Example 1) | | |

<Flatness Under AFM (Atomic Force Microscope)>

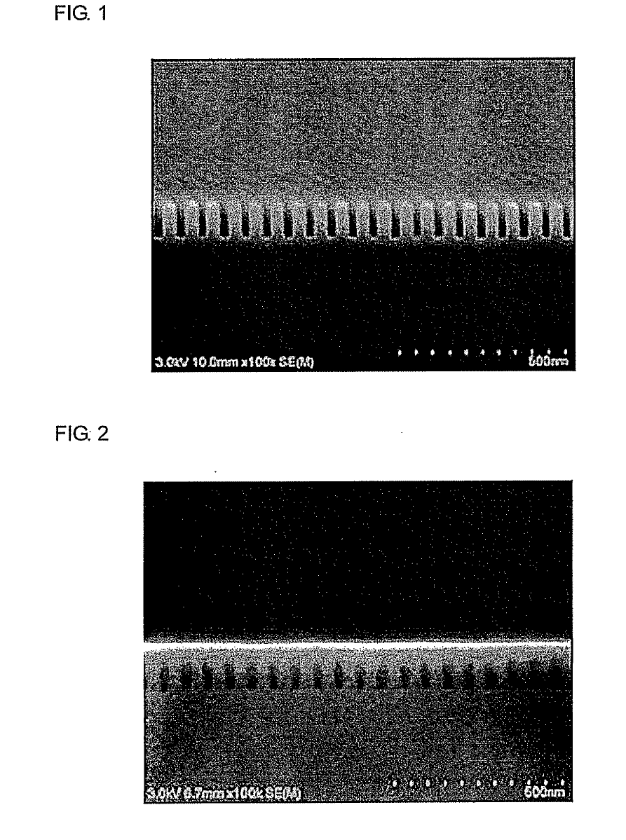

ASV-1 prepared in Example 1 was applied onto a substrate with structure (FIG. 1) by spin coating. The substrate with structure used was made of silicon, had a depth of 100 nm, and had lines and spaces which were equally spaced by 30 nm. The spin coating was carried out in the condition in which a film having a thickness of 85 nm could be formed on a silicon substrate without structure.

After spin coating, the film was exposed to light using an UV irradiation apparatus at an illumination intensity of 20 mW/cm² for 10 seconds under atmospheric pressure. Next, the film was subjected to post-exposure bake under atmospheric pressure using a hot plate at 130° C. for 5 minutes. The film surface after baking was subjected to surface analysis under AFM. The observed area under AFM was a perpendicular section with lines and spaces. The result of the surface analysis under AFM revealed that the planarizing film surface on the level difference had a concave-convex shape with a size of 1 nm or smaller and had a maximum surface roughness (Rₘₐₓ) of 1.483 nm.

From ASV-10 prepared in Example 10, a film was formed using the same substrate in the same manner as the above and was observed under AFM. The result of the surface analysis under AFM revealed that the planarizing film surface on the level difference had a concave-convex shape with a size of 1 nm or smaller and had a maximum surface roughness (Rₘₐₓ) of 1.499 nm.

From ASV-17 prepared in Comparative Example 3, a film was formed using the same substrate in the same manner as the above and was observed under AFM. The result of the surface analysis under AFM revealed that the planarizing film surface on the level difference had a concave-convex shape with a size of 3 nm or larger and had a maximum surface roughness (Rₘₐₓ) of 4.908 nm.

From ASV-18 prepared in Comparative Example 4, a film was formed using the same substrate in the same manner as the above and was observed under AFM. The result of the surface analysis under AFM revealed that the planarizing film surface on the level difference had a concave-convex shape with a size of 3 nm or larger and had a maximum surface roughness (Rₘₐₓ) of 6.337 nm.

The results of the films of ASV-1 and ASV-10 revealed that a material that is in a liquid form at room temperature under atmospheric pressure and has a molecular weight of 300 g/moL or more has small volume contraction ratios at the time of exposure and at the time of baking and provides excellent flatness on the substrate with structure.

In contrast, the result of the film of ASV-17 revealed that a material that has a small volume contraction ratio but is in a solid form at room temperature under atmospheric pressure provides poor flatness and large surface roughness. The result of the film of ASV-18 revealed that a material that has no contraction ratio but is in a solid form at room temperature under atmospheric pressure provides poor flatness and large surface roughness.

### INDUSTRIAL APPLICABILITY

The present invention is applicable to a planarizing film-forming composition for a hard disk that is a non-magnetic filler (planarizing film-forming composition) used for a method for forming tracks alternately having magnetic areas and non-magnetic areas, is sufficiently filled into fine grooves on a magnetic material surface, and is required not to cause contraction in the filled parts at the time of photo-curing (at the time of exposure) and at the time of post-exposure baking and to a method for producing a hard disk using the composition.

1. A planarizing film-forming composition for a hard disk comprising:
at least one polyfunctional (meth)acrylate compound being in a liquid state at room temperature and atmospheric pressure and having a molecular weight of 300 to 10,000.

2. The planarizing film-forming composition for a hard disk according to claim 1, wherein the compound is a compound having 2 to 20 (meth)acrylate groups in the molecule.

3. The planarizing film-forming composition for a hard disk according to claim 1, wherein the compound has a molecular weight of 300 to 2,300.

4. The planarizing film-forming composition for a hard disk according to claim 1, further comprising a photopolymerization initiator.

5. A method for producing a hard disk comprising:
a first step of forming a concave-convex shape on a magnetic material surface;
a second step of covering the magnetic material surface having the concave-convex shape with the planarizing film-forming composition as claimed in claim 1; and
a third step of etching the covered magnetic material surface for planarization until the magnetic material surface is exposed.

6. The method for producing a hard disk according to claim 5, further comprising a fourth step of covering the planarized magnetic material surface with a hard substance.

7. The method for producing a hard disk according to claim 5, wherein in the first step, the concave-convex shape is formed by nanoimprinting.

8. The method for producing a hard disk according to claim 5, wherein in the third step, the planarization is performed by dry etching.

9. The method for producing a hard disk according to claim 6, wherein the hard substance used in the fourth step is diamond-like carbon.
